(19) United States
(12) Patent Application Publication
(10) Pub. No.: US 2005/0042261 A1
(43) Pub. Date: Feb. 24, 2005

(54) Effervescent personal cleansing articles

(75) Inventors: Erik John Hasenoehrl, Loveland, OH (US); Kenneth Eugene Kyte III, Lebanon, OH (US); David Michael McAtee, Mason, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/645,079

(22) Filed: Aug. 21, 2003

(51) Int. Cl. A61K 9/70

(52) U.S. Cl. 424/443

(57) ABSTRACT

The invention is for cleansing articles that contain an effervescent foaming composition in a storage stabilized dry form, the composition alternatively being applied for facial and body cleansing article. A kit including a package for storage of the cleansing articles.

### CROSS-REFERENCE TO RELATED APPLICATIONS

This application claims priority to U.S. application Ser. No. 10/236,832, filed Sep. 6, 2002, which is a divisional of U.S. Pat. No. 6,495,151, granted Dec. 17, 2002, which is a divisional of U.S. Pat. No. 6,280,757, granted Aug. 28, 2001, which is a continuation-in-part (CIP) of U.S. Pat. No. 6,153,208, granted Nov. 28, 2000, which is a CIP of U.S. Pat. No. 6,190,678, granted Feb. 20, 2001, which is a CIP of U.S. Ser. No. 09/065,991, filed Apr. 24, 1998, now abandoned, which is a CIP of U.S. Pat. No. 6,132,746, granted Oct. 17, 2000.

### FIELD OF THE INVENTION

The invention concerns a cleansing article containing an effervescent foaming composition in a storage-stabilized form, the article alternatively being applied for facial and body cleansing.

### BACKGROUND OF THE INVENTION

Classically the process of cleansing skin or other sheets has employed a surfactant composition. Sometimes an implement is used with such a composition. Articles such as pads serve a multi-purpose including acting as delivery vehicle for the surfactant. Articles may also assist in generating foam and have exfoliating surfaces to assist in the cleansing function.

An early example of cleansing pad technology is found in U.S. Pat. No. 1,808,834 (Busch Sr.). A fabric pouch is disclosed surrounding a cleansing composition mainly consisting of calcium and sodium carbonate.

U.S. Pat. No. 4,234,442 (Cornelissens) describes an article that can consist of a water permeable material filled with an acidic and an alkaline constituent. Adipic, succinic and glutaric acids exemplify the acidic constituent. Sodium bicarbonate and carbonate represent the alkaline ingredient.

U.S. Pat. No. 4,272,393 (Gergely) describes a cleaning article formed of a porous flexible sheet, especially a cellulosic paper, impregnated with detergent and a gas-generating system. The latter is formed by separating an acidic component such as citric acid from a basic component such as sodium carbonate in two separate areas of the sheet.

U.S. Pat. No. 4,515,703 (Haq), U.S. Pat. No. 4,600,620 (Lloyd et al.), and U.S. Pat. No. 4,603,069 (Haq et al.) all describe wiping articles impregnated with surfactant. These do not contain any effervescent ingredients.

WO 97/43366 (Askew et al.) reports an effervescent system to improve dispensability of granular laundry detergent powders into wash water of automatic washing machines. Citric acid and bicarbonate combinations are employed to generate effervescence.

WO 99/48469 (Yagnik et al.) discloses powdered compositions some of which may be delivered via a tea bag. These compositions are formulated with an exothermic ingredient, a pH adjusting agent such as citric acid, optionally sodium bicarbonate for an effervescent effect and an aromatic ingredient for fragrance.

U.S. Pat. No. 6,063,390 (Farrell et al) discloses articles for cleansing body surfaces that is in the form of a envelope that is water-permeable wherein within the envelope is an effervescent cleansing composition in the form of an anhydrous dry solid comprising alkaline and acidic materials to generate gas and in combination with a solid surfactant for generating foam.

U.S. patent application Ser. No. 2002/0037255A1 (Charamubura et al) discloses envelope-like articles as disclosed in Farrell above that are sealed within an outer package formed of a film having breathability for carbon dioxide.

U.S. patent application Ser. No. 2003/0059387A1 (Bergquist) discloses envelope-like articles as disclosed by Farrell above wherein in addition to the alkaline and acid is a flowable particulate powder comprising a carrier, a skin benefit agent absorbed onto the carrier.

U.S. patent application Ser. No. 2003/0064042A1, (Bergquist), discloses envelope-like articles comprising effervescent cleansing composition capable of generating a foam upon contact with water; and a envelope formed of first and second water insoluble sheets, at least one being water permeable, the first and second sheets forming there between an area housing the cleansing composition, the second substrate comprising a non-woven sheet selected from the group consisting of meltblown, spunbond and sheet combinations.

U.S. patent application Ser. No. 2003/0063136A1, (Bergquist), discloses A curvilinear envelope wherein the hydroentangled fibers are entangled in a direction perpendicular to a major longitudinal axis of the web; and an effervescent cleanser composition.

U.S. patent No. 6,506,713 B1, (Slavtcheff et al) discloses envelope-like articles like Farrell above wherein the solid surfactant is replaced by dried plant solids.

Common to those of the aforementioned systems is the employment of effervescent anhydrous powders within the article for generating gas and foam when immersed in water.

Accordingly, it is an object of the present invention to eliminate dry flowing powders comprising the gas generating as well as a surfactant placed in a position other than with the gas generating chemistry wherein aggregating or caking of said chemistry inhibits generation of gas and transfer of water with surfactant to create optimum foaming. Still a further object of the invention is to provide a cleansing article that imparts a pleasant sensory feel to skin during and after use.

It is to be noted that the subsequently described invention is broader than the objects or technical problems it is directed to solve.

### SUMMARY OF THE INVENTION

A cosmetic article for cleansing body surfaces, the article comprising:
a) an effervescent composition comprising solid alkaline and acidic materials; and
b) a liquid-permeable laminate comprising a plurality of layers, said laminate having a first layer comprising a web and a second layer, in contact with said first layer, comprising a plurality of webs wherein said effervescent composition is present between the webs comprising said second layer.

Also provided is a method for cleansing skin involving wetting with water the article containing the effervescent composition delineated above, generating foam from the article and contacting skin surfaces with the generated foam. Further provided is a kit comprising said articles in a container designed to prevent loss of the effervescence of said article during storage and use that can be resealed after initial use of an article thereby deterring premature activation of effervescence of the articles remaining within said in said package.

### SUMMARY OF FIGURES

FIG. 1: Overhead planar view of article of the present invention.

FIG. 2: Side view of the article of the present invention.

FIG. 3: Cross-sectional view of a two layer laminate structure of the present invention.

FIG. 4: Cross-sectional view of a three layer laminate structure of the present invention.

FIG. 5: Cross-sectional view of a four layer laminate structure of the present invention.

### DETAILED DESCRIPTION OF THE INVENTION

Now it has been found that cleansing articles comprising a laminate structure with alkaline material and acid material located about the contacting surfaces of at plurality of webs comprising a second layer improves the stability of the cleansing article. The combined alkaline and acid materials do not aggregation or cake providing even and sustained reactivity of the chemistry during use. Furthermore, surfactants may be located at the surfaces of at least said first layer and other designated layers of the laminate for optimizing the foaming of the article to provide improved cleansing benefits. When the term “upon the web” is used, it is meant that the designated material is either on some or the entire surface of the web and, or within the interstices of the web itself. The layers of the article of the present comprise woven, non-woven and, or combination of woven and non-woven webs. The only requirement is that at least one of webs comprising a layer of the laminate be water-permeable to the extent that sufficient water passes into the article in order to activate the effervescing composition found within the laminate.

Laminate's Layers

Without being limited by theory, the laminate comprises a plurality of layers that in turn comprise a plurality of webs. These webs can enhance cleansing of the skin by facilitating lathering and exfoliation of the skin. By physically coming into contact with the skin or hair, the webs significantly aid in cleansing and removal of dirt, makeup, dead skin, and other debris. The webs can have the same or differing textures on each side. In preferred personal care embodiments, however, the webs are. non-scouring or nonabrasive to the skin.

The webs may comprise a variety of both natural and synthetic fibers or materials. As used herein, “natural fibers” are those derived from plants, animals, insects or byproducts of plants, animals, and insects. The conventional base starting material is usually a fibrous web comprising any of the common synthetic or natural textile-length fibers, or combinations thereof.

Nonlimiting examples of natural materials useful in the present invention include, but are not limited to, silk fibers, keratin fibers and cellulosic fibers. Nonlimiting examples of keratin fibers include those selected from the group consisting of wool fibers, camel hair fibers, and the like. Nonlimiting examples of cellulosic fibers include those selected from the group consisting of wood pulp fibers, cotton fibers, hemp fibers, jute fibers, flax fibers, and combinations thereof. Cellulosic fiber materials are preferred in the present invention. Nonlimiting examples of synthetic materials useful in the present invention include those selected from the group consisting of acetate fibers, acrylic fibers, cellulose ester fibers, modacrylic fibers, polyamide fibers, polyester fibers, polyolefin fibers, polyvinyl alcohol fibers, rayon fibers, formed films, films, and combinations thereof. Examples of suitable synthetic materials include acrylics such as acrilan, creslan, and the acrylonitrile-based fiber, orlon; cellulose ester fibers such as cellulose acetate, arnel, and acele; polyamides such as nylons (e.g., nylon 6, nylon 66, nylon 610, and the like); polyesters such as fortrel, kodel, and the polyethylene terephthalate fiber, polybutylene terephthalate fiber, dacron; polyolefins such as polypropylene, polyethylene; polyvinyl acetate fibers; polyurethane foams and combinations thereof. These and other suitable fibers and the nonwovens prepared there from are generally described in Riedel, “Nonwoven Bonding Methods and Materials,” Nonwoven World (1987); The Encyclopedia Americana, vol. 11, pp. 147-153, and vol. 26, pp. 566-581 (1984); U.S. Pat. No. 4,891,227, to Thaman et al., issued Jan. 2, 1990; and U.S. Pat. No. 4,891,228. As used herein, “nonwoven” means that the layer comprises fibers which are not woven into a fabric but rather are formed into a sheet, mat, or pad layer. The fibers can either be random (i.e., randomly aligned) or they can be carded (i.e., combed to be oriented in primarily one direction). Nonwoven substrates made from synthetic materials useful in the present invention can be obtained from a wide variety of commercial sources.

More preferred synthetic fibers for the sheet layer are solid staple polyester fibers, which comprise polyethylene terephthalate homopolymers. Suitable synthetic materials may include solid single component and multicomponent synthetic fibers, i.e., more than one type of material making up the fibers. The synthetic fibers may comprise bicomponent or dual component fibers. Such bicomponent fibers may have a core and a sheath configuration or a side-by-side configuration. In either instance, the sheet layer may comprise either a combination of fibers comprising the above-listed materials or fibers which themselves comprise a combination of the above-listed materials.

In any instance, side-by side configuration or core-sheath configuration, the fibers of the sheet layer may exhibit a helical or spiral configuration, particularly the bicomponent type fibers.

A preferred synthetic material for a scouring sheet layer may comprise nylon fibers. A more preferred synthetic material comprises nylon fibers formed into a scrim layer having additional nylon fibers bonded thereto such that the additional fibers form arcs on the scrim layer.

Natural material nonwovens useful in the present invention may be obtained from a wide variety of commercial sources. Nonlimiting examples of suitable commercially available paper layers useful herein include Airtex®, an embossed airlaid cellulosic layer having a base weight of about 71 gsy, available from James River, Green Bay, Wis.; and Walkisoft®, an embossed airlaid cellulosic having a base weight of about 75 gsy, available from Walkisoft U.S.A., Mount Holly, N.C.

Additional suitable nonwoven materials include, but are not limited to, those disclosed in U.S. Pat. No. 4,447,294, issued to Osborn on May 8, 1984; U.S. Pat. No. 4,603,176 issued to Bjorkquist on Jul. 29, 1986; U.S. Pat. No. 4,981,557 issued to Bjorkquist on Jan. 1, 1991; U.S. Pat. No. 5,085,736 issued to Bjorkquist on Feb. 4, 1992; U.S. Pat. No. 5,138,002 issued to Bjorkquist on Aug. 8, 1992; U.S. Pat. No. 5,262,007 issued to Phan et al. on Nov. 16, 1993; U.S. Pat. No. 5,264,082, issued to Phan et al. on Nov. 23, 1993; U.S. Pat. No. 4,637,859 issued to Trokhan on Jan. 20, 1987; U.S. Pat. No. 4,529,480, issued to Trokhan on Jul. 16, 1985; U.S. Pat. No. 4,687,153 issued to McNeil on Aug. 18, 1987; U.S. Pat. No. 5,223,096, issued to Phan et al. on Jun. 29, 1993 and U.S. Pat. No. 5,679,222, issued to Rasch et al. on Oct. 21, 1997.

Additional suitable materials include but are not limited to, formed films and composite materials, i.e., multiply materials containing formed films. Alternatively, such formed films comprise plastics which tend to be soft to the skin. Suitable soft plastic formed films include, but are not limited to, polyolefins such as low density polyethylenes (LDPE).

Methods of making nonwovens are well known in the art. Generally, these nonwovens can be made by air-laying, water-laying, meltblowing, coforming, spunbonding, or carding processes in which the fibers or filaments are first cut to desired lengths from long strands, passed into a water or air stream, and then deposited onto a screen through which the fiber-laden air or water is passed. The resulting layer, regardless of its method of production or composition, is then subjected to at least one of several types of bonding operations to anchor the individual fibers together to form a self-sustaining web. In the present invention the nonwoven layer can be prepared by a variety of processes including, but not limited to, meltblowing, spunbonding, air-entanglement, hydroentanglement, thermal bonding, and combinations of these processes.

Laminate's First Layer

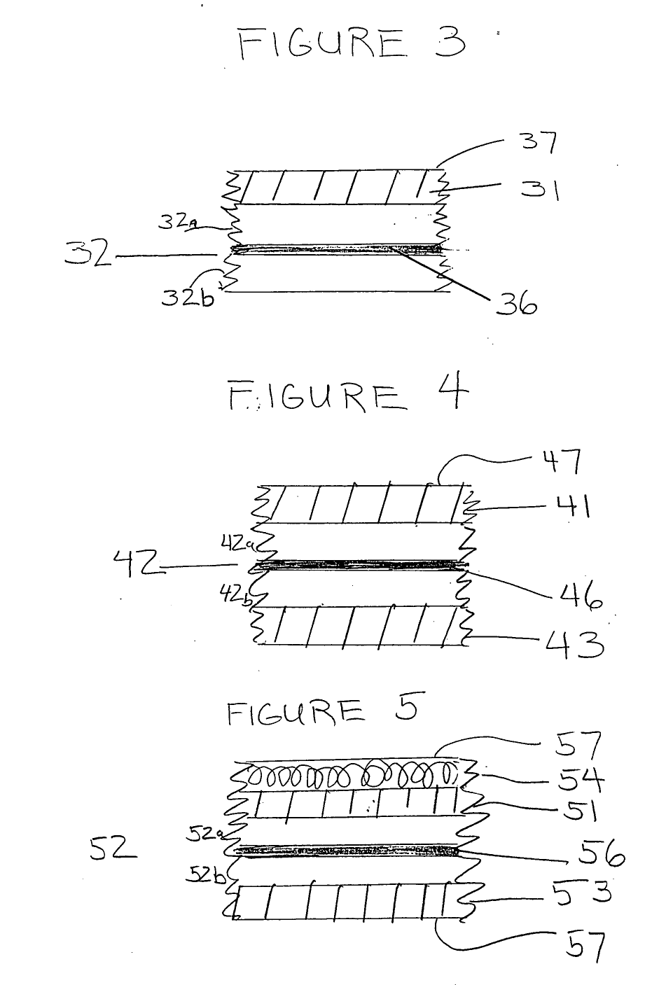

The first layer of the laminate making up the article of the present invention can comprise any of the materials discussed above. As shown in FIG. 3, the first layer 31 is in communication over its entire surface with the top surface of a second layer 32. These two layers are joined to form the laminate by routine methods known in the art. This first layer is typically designed to contact the surface of the skin to be cleansed and has certain desirable physical characteristics for the particular purpose intended. For example, the first layer may be that so that it is soft to treat sensitive skin, or coarse enough to facilitate skin exfoliation. FIGS. 4 and 5 illustrate embodiment wherein an additional layer 43 and 53 respectively are applied to layers 42 and 52 respectively at the surfaces opposite the contacting surfaces of 41-42 and 51-52 respectively.

Laminate's Second Layer

The second layer of the laminate of the present article contains the effervescent composition is present between the webs comprising the second layer. This composition is applied to portions of or the entire surface of said webs comprising said second layer forming itself a laminate structure within the overall laminate structure of the article. By placing the effervescing composition between the webs of the second layer, the effervescent composition does not agglomeration and, or cake, thereby remaining evenly spread over the web surface to throughout the laminate and facilitating consistent and steady activity of the article when wetted.

Embodiment as illustrated in FIG. 3 is one wherein the second layer 32 comprises two webs 32a and 32b, wherein the effervescent composition 36 is placed between webs 32a and 32b using a process similar to that disclosed in U.S. Pat. No. 6,093,474 issued Jul. 25, 2000 and U.S. patent application Ser. No. 2003/0084994 A1. Whether the second layer contains two webs as shown in the FIGURES or a plurality greater than two webs, the effervescent composition as illustrated in FIGS. 3, 4 and 5 as 36, 46 and 56 respectively, is either added separately to the web or as a homogeneous premix blend, in a dry state. This effervescent composition is distributed over the contact surfaces between webs comprising said second layer. It is preferred that the effervescent material be evenly distributed over the entire contact surface between the webs comprising the second layer.

The process for making the second layer involves feeding the effervescent composition onto the web from a depositing station that comprises one or more dispensers or hoppers that feed the effervescent materials individually or the composition as a whole onto the contact surfaces between the all or some of the webs comprising the second layer. Upon deposition onto the web surface, the effervescent composition adheres to the web using any number of means including a bonding agent either applied to the web prior to or along with the effervescent composition. Bonding agents useful in the manufacture of the second layer includes, but is not limited to thermoplastic polymers including polypropylene, polyethylene, ethylene vinyl acetate or other synthetic or natural bonding agent. The amount of the bonding age is from 3% to 30% by weight of the effervescent composition. The blending of the effervescent materials forming the composition and bonding agent takes place in a mixer fed by suitable flow rates of the substances through tubes wherein the mixer in turn feeds into dispensers a tube.

After passing through a means for activating the bonding agent, such as a heating device, the web can have a second web applied over the top of the first web. The first web has an adhesive applied to it wherein a second web is applied over the first web. After passing through a series of rollers, additional webs with or without the effervescent composition are laminated into the second layer in the manner previously disclosed. Although not necessary, the effervescent composition may also be applied at the contacting surfaces between the first and the second layers forming the laminate.

Effervescent Compositions

The effervescent compositions of the present include acidic materials. Suitable for this purpose are any acids present in dry solid form. Especially appropriate are C₂ to C₂₀ organic mono- and poly-carboxylic acids and especially alpha- and beta-hydroxycarboxylic acids; C₂ to C₂₀ organophosphorus acids such as phytic acid; C₂ to C₂₀ organosulfur acids such as toluene sulfonic acid; and peroxides such as hydrogen peroxide. Typical hydroxycarboxylic acids include adipic, glutaric, succinic, tartaric, malic, maleic, lactic, salicylic and citric acids as well as acid forming lactones such as gluconolactone and glucarolactone. Most preferred is citric acid. Also suitable as acid material may be encapsulated acids. Typical encapsulating material may include water-soluble synthetic or natural polymers such as polyacrylates (e.g. encapsulating polyacrylic acid), cellulosic gums, polyurethane and polyoxyalkylene polymers. By the term “acid” is meant any substance which when dissolved in deionized water at 1% concentration will have a pH of less than 7, alternatively less than 6.5, optimally less than 5. These acids alternatively at 25° C. are in solid form, i.e. having melting points no less than 25° C. Concentrations of the acid should range from about 0.5% to about 80%, alternatively from about 10% to about 65%, and finally from about 20% to about 45% by weight of the total composition.

Another important component of the effervescent compositions is alkaline materials. The alkaline materials are substances that can generate a gas such as carbon dioxide, nitrogen or oxygen, i.e. effervesce, when contacted with water and the acidic material. Suitable alkaline materials are selected from the group consisting of anhydrous salts of carbonates and bicarbonates, alkaline peroxides (e.g. sodium perborate and sodium percarbonate) and azides (e.g. sodium azide) and mixtures thereof. Alternatively, the alkaline materials are sodium or potassium bicarbonate. Amounts of the alkaline materials may range from about 1% to about 80%, alternatively from about 5% to about 49%, alternatively from about 15% to about 40%, and finally from about 25% to about 35% by weight of the total composition.

By the term “anhydrous” is meant the presence of no more than 5%, alternatively no more than 3.5% and optimally no more than 1% water by weight of the total composition. Water of hydration is not considered to be water for purposes of the anhydrous definition. However, it is preferred to minimize, alternatively to eliminate any water of hydration. Advantageously the combined amount of acidic and alkaline materials will be at least about 1.5%, alternatively from about 40% to about 95%, and finally from about 60% to about 80% by weight of the total composition.

Film Formed Materials

Formed films may be an additional layer of the laminate structure of the present invention. The film formed may provide the article with a laminate that has rigidity, to prevent balling up of the article when being wiped over the surface of the body. This is particularly helpful for larger surface area sheets such as those typically found in body wash articles as described in U.S. Pat. No. 6,491,928, Smith et al, issued Dec. 10, 2002. The formed films also assist in constructing a spot fused texture over the planar surface of the article, reminiscent of a quilted mattress. Such a pattern again assists in maintaining in use rigidity and also equal distribution of any lose dry, granular materials that may be distributed between the layers of the laminate. Formed films include, but are not limited to polymer films such as polyolefins including polyethylene.

As previously discussed, the laminate must have at least a layer that is water permeable. Thus, the formed films may also be water-permeable. One such way to insure permeability is to create aperatured formed films. Aperatured formed films may be apertured, microaperatured, macroaperatured and combinations thereof. On the other hand, “macroapertured”, as used herein refers to a layer containing well-defined apertures having an average diameter of greater than about 300 microns. As used herein, “microapertured” generally refers to layers containing well-defined microscopic apertures (i.e., those not readily visible to the naked eye having 20/20 vision). Preferably, the microapertures are characterized by the presence of petal-like edged surface aberrations on at least one surface of the layer which add a three dimensional character to the layer. Preferably, the microapertures are characterized by the presence of petal-like edged surface aberrations on at least one surface of the layer such that the layer has a three dimensional character and such that fluid flow is facilitated from one surface of the layer to another surface of the layer. Suitable microapertured materials useful for the first layer of the present invention include, but are not limited to, those disclosed in co-pending application U.S. Ser. No. 08/326,571 and PCT Application No. US95/07435, filed Jun. 12, 1995 and published Jan. 11, 1996, and U.S. Pat. No. 4,629,643, issued on Dec. 16, 1986 to Curro et al., which are incorporated by reference herein in their entirety.

High Loft Batting Materials

A high loft batting layer may also be a part of the laminate of the present invention. FIG. 5 illustrates such a layer 54. This batting is lofty, non-scouring, and is of low-density. Such materials are described is U.S. Pat. No. 6,428,799, issued Aug. 6, 2002. As used herein, “lofty” means that the layer has density of from about 0.00005 g/cm³ to about 0.1 g/cm³, preferably from about 0.001 g/cm³ to about 0.09 g/cm³ and a thickness of from about 0.04 inches to about 2 inches at 5 gms/in². Also, as used herein, “non-scouring” means having an Abrasiveness Value of greater than about 15, preferably greater than about 30, more preferably great than about 50, even more preferably greater than about 70, and most preferably greater than about 80, as defined by the Abrasiveness Value Methodology described below.

The batting layer preferably comprises synthetic materials. As used herein, “synthetic” means that the materials are obtained primarily from various man-made materials or from natural materials that have been further altered. Suitable synthetic materials include, but are not limited to, acetate fibers, acrylic fibers, cellulose ester fibers, modacrylic fibers, polyamide fibers, polyester fibers, polyolefin fibers, polyvinyl alcohol fibers, rayon fibers, polyethylene foam, polyurethane foam, and combinations thereof. Preferred synthetic materials, particularly fibers, may be selected from the group consisting of nylon fibers, rayon fibers, polyolefin fibers, polyester fibers, and combinations thereof. Preferred polyolefin fibers are fibers selected from the group consisting of polyethylene, polypropylene, polybutylene, polypentene, and combinations and copolymers thereof. Suitable synthetic materials may include solid single component (i.e., chemically homogeneous) fibers, multiconstituent fibers (i.e., more than one type of material making up each fiber), and multicomponent fibers (i.e., synthetic fibers which comprise two or more distinct filament types which are somehow intertwined to produce a larger fiber), and combinations thereof. Preferred fibers include bicomponent fibers, multiconstituent fibers, and combinations thereof. Such bicomponent fibers may have a core-sheath configuration or a side-by-side configuration. In either instance, the batting layer may comprise either a combination of fibers comprising the above-listed materials or fibers which themselves comprise a combination of the above-listed materials.

The batting layer may also comprise natural fibers. Suitable natural fibers are described below in the Nonwoven Layer section.

Furthermore, the fibers of the batting layer preferably have an average thickness of from about 0.5 microns to about 150 microns.

In another embodiment, the batting layer of the present invention may comprise a composite material, i.e., a material having one or more plies of the same or different suitable materials merely superimposed physically, joined together continuously (e.g., laminated, etc.) or in a discontinuous pattern, or by bonding at the external edges (or periphery) of the layer and/or at discrete loci.

Without being limited by theory, the batting layer is especially useful as a skin contact side in the personal care article of the present invention due to its mild exfoliating properties. Furthermore, the preferred synthetic fibers which make up the batting layer are largely hydrophobic thus allowing water to readily flow through the article to reach the cleansing component and readily flow out from the article to produce a high volume of flash lather which tapers off during use but which still suffices through an entire showering or bathing experience. In an embodiment of the present invention, surfactants are applied to the batting material using routine coating methods known in the art and found in commercial art.

Lathering Characteristics of the Article

As previously stated, the objective of the article of the present invention is as a skin cleansing product, therefore considered as a “lathering product” or “lathering article”. As such, the article comprises selected surfactants to achieve optimum skin cleansing. The surfactants are located at one or more poin6ts within or upon the laminate. In FIG. 3, the surfactants are primarily located on the first layer on the surface of the web opposite the contacting surfaces of said first and second layers of the laminate. FIG. 4 illustrates placing the surfactants at the surfaces of the laminate 47. FIG. 5 illustrates surfactant 57 at both the surface of batting layer 54 opposite the surface of the batting layer contacting layer 51 and at the lower layer 55 opposite the contact surfaces of layers 51 and 52. The surfactants can also be located with the effervescent composition 36, 46 and 56 of FIGS. 3, 4 and 5. Furthermore, the surfactants can be located in a combination of places including all those disclosed above.

Surfactants, particularly those characterized as lathering surfactants are used in the present invention to provide easy and rapid foam generation at surfactants levels desirable to provide skin mildness of the article. Generally the articles will contain surfactants at a level from about 0.01% to 1500%, by weight of the substrate. Alternatively, the articles of the present invention comprise surfactants at levels from about 0.05% to about 100%, alternatively from about 0.06% to about 50%, and finally from about 0.07% to about 25%, based on the weight of the water insoluble sheets.

By a “lathering surfactant” is meant a surfactant, that when combined with water and mechanically agitated generates a foam or lather. Alternatively, these surfactants should be mild, which means that these surfactants provide sufficient cleansing or detersive benefits but do not overly dry the skin or hair (e.g., removing too much natural oil and/or moisture), while still meeting the lathering criteria described above. The present invention comprises one or more lathering surfactants that are releasably associated with the water-insoluble substrate. Thus the lathering surfactants can be added onto or impregnated into the webs making up the respective layer of the article. This is preferably done prior to the point of assembly or use of the article, i.e., the surfactants will be combined with the webs comprising said layers of said article wherein the article is dried prior to its being packaged an ultimately wetted for use. Preferred articles of the present invention comprise a sufficient amount of one or more lathering surfactants such that the articles are capable of generating ≧30 ml of Lather Volume (medium hardness water at 95° C.) according to the Lather Volume Test described below.

A wide variety of lathering surfactants are useful herein and include those selected from the group consisting of anionic lathering surfactants, nonionic lather surfactants, amphoteric lathering surfactants, and mixtures thereof. Generally, the lathering surfactants do not strongly interfere with deposition of any skin care active and, or conditioning agents that are present, e.g., are fairly water soluble, and usually have an HLB value of above 10. Cationic surfactants can also be used as optional components, provided they do not negatively impact the overall lathering characteristics of the required lathering surfactants.

Anionic Lathering Surfactants

Nonlimiting examples of anionic lathering surfactants useful in the compositions of the present invention are disclosed in McCutcheon's, Detergents and Emulsifiers, North American edition (1986), published by allured Publishing Corporation; McCutcheon's, Functional Materials, North American Edition (1992); and U.S. Pat. No. 3,929,678, to Laughlin et al., issued Dec. 30, 1975, all of which are incorporated by reference herein in their entirety.

A wide variety of anionic lathering surfactants are useful herein. Nonlimiting examples of anionic lathering surfactants include those selected from the group consisting of sarcosinates, sulfates, isethionates, taurates, phosphates, lactylates, glutamates, and mixtures thereof. Amongst the isethionates, the alkoyl isethionates are preferred, and amongst the sulfates, the alkyl and alkyl ether sulfates are preferred. The alkoyl isethionates typically have the formula RCO—OCH₂CH₂SO₃M wherein R is alkyl or alkenyl of from about 10 to about 30 carbon atoms, and M is a water-soluble cation such as ammonium, sodium, potassium and triethanolamine. Nonlimiting examples of these isethionates include those alkoyl isethionates selected from the group consisting of ammonium cocoyl isethionate, sodium cocoyl isethionate, sodium lauroyl isethionate, and mixtures thereof.

The alkyl and alkyl ether sulfates typically have the respective formulas ROSO₃M and RO(C₂H₄O)ₓSO₃M, wherein R is alkyl or alkenyl of from about 10 to about 30 carbon atoms, x is from about 1 to about 10, and M is a water-soluble cation such as ammonium, sodium, potassium and triethanolamine. Another suitable class of anionic surfactants are the water-soluble salts of the organic, sulfuric acid reaction products of the general formula:
R1-SO₃-M
wherein R1 is chosen from the group consisting of a straight or branched chain, saturated aliphatic hydrocarbon radical having from about 8 to about 24, alternatively about 10 to about 16, carbon atoms; and M is a cation. Still other anionic synthetic surfactants include the class designated as succinamates, olefin sulfonates having about 12 to about 24 carbon atoms, and b-alkyloxy alkane sulfonates. Examples of these materials are sodium lauryl sulfate and ammonium lauryl sulfate.

Other anionic materials useful herein are soaps (i.e., alkali metal salts, e.g., sodium or potassium salts) of fatty acids, typically having from about 8 to about 24 carbon atoms, alternatively from about 10 to about 20 carbon atoms. The fatty acids used in making the soaps can be obtained from natural sources such as, for instance, plant or animal-derived glycerides (e.g., palm oil, coconut oil, soybean oil, castor oil, tallow, lard, etc.) The fatty acids can also be synthetically prepared. Soaps are described in more detail in U.S. Pat. No. 4,557,853, cited above.

Other anionic materials include phosphates such as monoalkyl, dialkyl, and trialkylphosphate salts.

Other anionic materials include alkanoyl sarcosinates corresponding to the formula RCON(CH₃)CH₂CH₂CO₂M wherein R is alkyl or alkenyl of about 10 to about 20 carbon atoms, and M is a water-soluble cation such as ammonium, sodium, potassium and alkanolamine (e.g., triethanolamine), a preferred examples of which are sodium lauroyl sarcosinate, sodium cocoyl sarcosinate, ammonium lauroyl sarcosinate, and sodium myristoyl sarcosinate. TEA salts of sarcosinates are also useful.

Also useful are taurates that are based on taurine, which is also known as 2-aminoethanesulfonic acid. Especially useful are taurates having carbon chains between C₈ and C₁₆. Examples of taurates include N-alkyltaurines such as the one prepared by reacting dodecylamine with sodium isethionate according to the teaching of U.S. Pat. No. 2,658,072 which is incorporated herein by reference in its entirety. Further nonlimiting examples include ammonium, sodium, potassium and alkanolamine (e.g., triethanolamine) salts of lauroyl methyl taurate, myristoyl methyl taurate, and cocoyl methyl taurate.

Also useful are lactylates, especially those having carbon chains between C₈ and C₁₆. Nonlimiting examples of lactylates include ammonium, sodium, potassium and alkanolamine (e.g., triethanolamine) salts of lauroyl lactylate, cocoyl lactylate, lauroyl lactylate, and caproyl lactylate.

Also useful herein as anionic surfactants are glutamates, especially those having carbon chains between C₈ and C₁₆. Nonlimiting examples of glutamates include ammonium, sodium, potassium and alkanolamine (e.g., triethanolamine) salts of lauroyl glutamate, myristoyl glutamate, and cocoyl glutamate.

Nonlimiting examples of preferred anionic lathering surfactants useful herein include those selected from the group consisting of sodium lauryl sulfate, ammonium lauryl sulfate, ammonium laureth sulfate, sodium laureth sulfate, sodium trideceth sulfate, ammonium cetyl sulfate, sodium cetyl sulfate, ammonium cocoyl isethionate, sodium lauroyl isethionate, sodium lauroyl lactylate, triethanolamine lauroyl lactylate, sodium caproyl lactylate, sodium lauroyl sarcosinate, sodium myristoyl sarcosinate, sodium cocoyl sarcosinate, sodium lauroyl methyl taurate, sodium cocoyl methyl taurate, sodium lauroyl glutamate, sodium myristoyl glutamate, and sodium cocoyl glutamate and mixtures thereof.

Especially preferred for use herein is ammonium lauryl sulfate, ammonium laureth sulfate, sodium lauroyl sarcosinate, sodium cocoyl sarcosinate, sodium myristoyl sarcosinate, sodium lauroyl lactylate, and triethanolamine lauroyl lactylate.

Nonionic Lathering Surfactants

Nonlimiting examples of nonionic lathering surfactants for use in the compositions of the present invention are disclosed in McCutcheon's, Detergents and Emulsifiers, North American edition (1986), published by allured Publishing Corporation; and McCutcheon's, Functional Materials, North American Edition (1992); both of which are incorporated by reference herein in their entirety.

Nonionic lathering surfactants useful herein include those selected from the group consisting of alkyl glucosides, alkyl polyglucosides, polyhydroxy fatty acid amides, alkoxylated fatty acid esters, lathering sucrose esters, amine oxides, and mixtures thereof.

Alkyl glucosides and alkyl polyglucosides are useful herein, and can be broadly defined as condensation articles of long chain alcohols, e.g. C₈₋₃₀ alcohols, with sugars or starches or sugar or starch polymers, i.e., glycosides or polyglycosides. These compounds can be represented by the formula (S)ₙ—O—R wherein S is a sugar moiety such as glucose, fructose, mannose, and galactose; n is an integer of from about 1 to about 1000, and R is a C₈₋₃₀ alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants include those wherein S is a glucose moiety, R is a C₈₋₂₀ alkyl group, and n is an integer of from about 1 to about 9. Commercially available examples of these surfactants include decyl polyglucoside (available as APG 325 CS from Henkel) and lauryl polyglucoside (available as APG 600CS and 625 CS from Henkel). Also useful are sucrose ester surfactants such as sucrose cocoate and sucrose laurate.

Other useful nonionic surfactants include polyhydroxy fatty acid amide surfactants, more specific examples of which include glucosamides, corresponding to the structural formula: 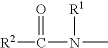
wherein: R¹ is H, C₁-C₄ alkyl, 2-hydroxyethyl, 2-hydroxy-propyl, alternatively C₁-C₄ alkyl, more alternatively methyl or ethyl, most alternatively methyl; R² is C₅-C₃₁ alkyl or alkenyl, alternatively C₇-C₁₉ alkyl or alkenyl, more alternatively C₉-C₁₇ alkyl or alkenyl, most alternatively C₁₁-C₁₅ alkyl or alkenyl; and Z is a polhydroxyhydrocarbyl moiety having a linear hydrocarbyl chain with a least 3 hydroxyls directly connected to the chain, or an alkoxylated derivative (alternatively ethoxylated or propoxylated) thereof. Z alternatively is a sugar moiety selected from the group consisting of glucose, fructose, maltose, lactose, galactose, mannose, xylose, and mixtures thereof. An especially preferred surfactant corresponding to the above structure is coconut alkyl N-methyl glucoside amide (i.e., wherein the R²CO— moiety is derived from coconut oil fatty acids). Processes for making compositions containing polyhydroxy fatty acid amides are disclosed, for example, in G. B. Patent Specification 809,060, published Feb. 18, 1959, by Thomas Hedley & Co., Ltd.; U.S. Pat. No. 2,965,576, to E. R. Wilson, issued Dec. 20, 1960; U.S. Pat. No. 2,703,798, to A. M. Schwartz, issued Mar. 8, 1955; and U.S. Pat. No. 1,985,424, to Piggott, issued Dec. 25, 1934; which are incorporated herein by reference in their entirety.

Other examples of nonionic surfactants include amine oxides. Amine oxides correspond to the general formula R¹R²R³NO, wherein R¹ contains an alkyl, alkenyl or monohydroxy alkyl radical of from about 8 to about 18 carbon atoms, from 0 to about 10 ethylene oxide moieties, and from 0 to about 1 glyceryl moiety, and R² and R³ contain from about 1 to about 3 carbon atoms and from 0 to about 1 hydroxy group, e.g., methyl, ethyl, propyl, hydroxyethyl, or hydroxypropyl radicals. The arrow in the formula is a conventional representation of a semipolar bond. Examples of amine oxides suitable for use in this invention include dimethyl-dodecylamine oxide, oleyldi(2-hydroxyethyl) amine oxide, dimethyloctylamine oxide, dimethyl-decylamine oxide, dimethyl-tetradecylamine oxide, 3,6,9-trioxaheptadecyldiethylamine oxide, di(2-hydroxyethyl)-tetradecylamine oxide, 2-dodecoxyethyldimethylamine oxide, 3-dodecoxy-2-hydroxypropyldi(3-hydroxypropyl)amine oxide, dimethylhexadecylamine oxide.

Nonlimiting examples of preferred nonionic surfactants for use herein are those selected form the group consisting of C₈-C₁₄ glucose amides, C₈-C₁₄ alkyl polyglucosides, sucrose cocoate, sucrose laurate, lauramine oxide, cocoamine oxide, and mixtures thereof.

Amphoteric Lathering Surfactants

The term “amphoteric lathering surfactant,” as used herein, is also intended to encompass zwitterionic surfactants, which are well known to formulators skilled in the art as a subset of amphoteric surfactants.

A wide variety of amphoteric lathering surfactants can be used in the compositions of the present invention. Particularly useful are those which are broadly described as derivatives of aliphatic secondary and tertiary amines, alternatively wherein the nitrogen is in a cationic state, in which the aliphatic radicals can be straight or branched chain and wherein one of the radicals contains an ionizable water solubilizing group, e.g., carboxy, sulfonate, sulfate, phosphate, or phosphonate.

Nonlimiting examples of amphoteric surfactants useful in the compositions of the present invention are disclosed in McCutcheon's, Detergents and Emulsifiers, North American edition (1986), published by allured Publishing Corporation; and McCutcheon's, Functional Materials, North American Edition (1992), both of which are incorporated by reference herein in their entirety.

Nonlimiting examples of amphoteric or zwitterionic surfactants are those selected from the group consisting of betaines, sultaines, hydroxysultaines, alkyliminoacetates, iminodialkanoates, aminoalkanoates, and mixtures thereof.

Examples of betaines include the higher alkyl betaines, such as coco dimethyl carboxymethyl betaine, lauryl dimethyl carboxymethyl betaine, lauryl dimethyl alpha-carboxyethyl betaine, cetyl dimethyl carboxymethyl betaine, cetyl dimethyl betaine (available as Lonzaine 16SP from Lonza Corp.), lauryl bis-(2-hydroxyethyl) carboxymethyl betaine, oleyl di-methyl gamma-carboxypropyl betaine, lauryl bis-(2-hydroxypropyl)alpha-carboxyethyl betaine, coco dimethyl sulfopropyl betaine, lauryl dimethyl sulfoethyl betaine, lauryl bis-(2-hydroxyethyl) sulfopropyl betaine, amidobetaines and amidosulfobetaines (wherein the RCONH(CH₂)₃ radical is attached to the nitrogen atom of the betaine), oleyl betaine (available as amphoteric Velvetex OLB-50 from Henkel), and cocamidopropyl betaine (available as Velvetex BK-35 and BA-35 from Henkel).

Examples of sultaines and hydroxysultaines include materials such as cocamidopropyl hydroxysultaine (available as Mirataine CBS from Rhone-Poulenc).

Preferred for use herein are amphoteric surfactants having the following structure: 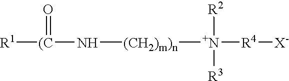
wherein R¹ is unsubstituted, saturated or unsaturated, straight or branched chain alkyl having from about 9 to about 22 carbon atoms. Preferred R¹ has from about 11 to about 18 carbon atoms; more alternatively from about 12 to about 18 carbon atoms; more alternatively still from about 14 to about 18 carbon atoms; m is an integer from 1 to about 3, more alternatively from about 2 to about 3, and more alternatively about 3; n is either 0 or 1, alternatively 1; R² and R³ are independently selected from the group consisting of alkyl having from 1 to about 3 carbon atoms, unsubstituted or mono-substituted with hydroxy, preferred R² and R³ are CH₃; X is selected from the group consisting of CO₂, SO₃ and SO₄; R⁴ is selected from the group consisting of saturated or unsaturated, straight or branched chain alkyl, unsubstituted or monosubstituted with hydroxy, having from 1 to about 5 carbon atoms. When X is CO₂, R⁴ alternatively has 1 or 3 carbon atoms, more alternatively 1 carbon atom. When X is SO₃ or SO₄, R⁴ alternatively has from about 2 to about 4 carbon atoms, more alternatively 3 carbon atoms.

Examples of amphoteric surfactants of the present invention include the following compounds:

Cetyl dimethyl betaine (this material also has the CTFA designation cetyl betaine) 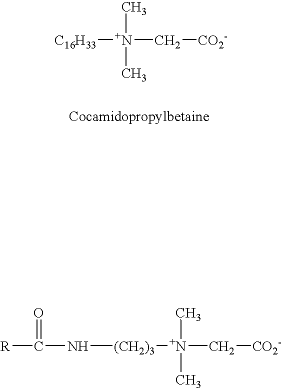
wherein R has from about 9 to about 13 carbon atoms

Cocamidopropyl hydroxy sultaine 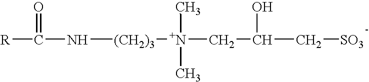
wherein R has from about 9 to about 13 carbon atoms,

Examples of other useful amphoteric surfactants are alkyliminoacetates, and iminodialkanoates and aminoalkanoates of the formulas RN[CH₂)ₘCO₂M]₂ and RNH(CH₂)ₘCO₂M wherein m is from 1 to 4, R is a C₈-C₂₂ alkyl or alkenyl, and M is H, alkali metal, alkaline earth metal ammonium, or alkanolammonium. Also included are imidazolinium and ammonium derivatives. Specific examples of suitable amphoteric surfactants include sodium 3-dodecyl-aminopropionate, sodium 3-dodecylaminopropane sulfonate, N-higher alkyl aspartic acids such as those produced according to the teaching of U.S. Pat. No. 2,438,091, which is incorporated herein by reference in its entirety; and the products sold under the trade name “Miranol” and described in U.S. Pat. No. 2,528,378, which is incorporated herein by reference in its entirety. Other examples of useful amphoterics include amphoteric phosphates, such as coamidopropyl PG-dimonium chloride phosphate (commercially available as Monaquat PTC, from Mona Corp.). Also useful are amphoacetates such as disodium lauroamphodiacetate, sodium lauroamphoacetate, and mixtures thereof.

Preferred lathering surfactants for use herein are the following, wherein the anionic lathering surfactant is selected from the group consisting of ammonium lauroyl sarcosinate, sodium trideceth sulfate, sodium lauroyl sarcosinate, ammonium laureth sulfate, sodium laureth sulfate, ammonium lauryl sulfate, sodium lauryl sulfate, ammonium cocoyl isethionate, sodium cocoyl isethionate, sodium lauroyl isethionate, sodium cetyl sulfate, sodium lauroyl lactylate, triethanolamine lauroyl lactylate, and mixtures thereof; wherein the nonionic lathering surfactant is selected from the group consisting of lauramine oxide, cocoamine oxide, decyl polyglucose, lauryl polyglucose, sucrose cocoate, C₁₂₋₁₄ glucosamides, sucrose laurate, and mixtures thereof; and wherein the amphoteric lathering surfactant is selected from the group consisting of disodium lauroamphodiacetate, sodium lauroamphoacetate, cetyl dimethyl betaine, cocoamidopropyl betaine, cocoamidopropyl hydroxy sultaine, and mixtures thereof.

As used herein, the terms means that the product or article contains enough of the surfactants described herein that it can generate ≧30 ml of Lather Volume, as described herein in the Lather Volume Test. These Lather Volume measurements are conducted with a medium hardness water (8-10 grains per gallon) at 95 C. The Lather Volume test described below shows the articles of the present invention comprise sufficient lathering surfactant wherein the articles can generate greater than or equal to about 30 ml, alternatively greater than or equal to about 50 ml, alternatively greater than or equal to about 75 ml, and finally greater than or equal to about 150 ml of Average Lather Volume.

The Average Lather Volume is a measurement determined by the Lather Volume Test. This test provides a consistent measurement of the volume of lather/foam generated by the articles described herein. The Lather Volume Test procedure is as follows:
(1) Wash hands with Ivory® soap bar before conducting the test thereby removing soils from hands that may affect the accuracy of the measurement.
(2) Holding the test article open in the non-dominant hand with the edges turned up, add via a 10 syringe or a Brinkmann re-pipetter 10 mls. of water at 95° C. having medium hardness of about 8-10 grains per gallon
(3) Generate lather by rubbing using medium pressure the test article with the dominant hand in a circular motion between the palms for 6 seconds (about 2 rotations per second), balling-up the article between the palms of the hand.
(4) Hold open in the non-dominant hand the article, adding onto the article 10 ml of water via a 10 cc syringe or a Brinkmann repipetterat 95° C. having medium hardness of about 8-10 grains per gallon. Again rub the article with the dominant had (about 3 rotations) using moderate force (e.g, 4 oz.) balling-up the test article between the palms.
(5) Open the test article and activate lathering by rubbing the article upon itself about 5 times by holding one edge of the article in one hand and rotating the hand holding the other side.
(6) Flip the test article over and repeat Step #6 rotating the article using the non-dominate hand.
(7) Gather lather off the test article, holding the test article in a cupped hand and scraping the its surface with the other hand, being careful to only scrape lather from the test article. Place the lather from the test article into a large graduated cylinder or beaker. Repeat this procedure 5 times on the same test article, collecting the lather from each iteration in the same graduated cylinder or beaker to measure the total accumulated lather from these iterations. This level is defined as as the Lather Volume.

To achieve consistent results, the Average Lather Volume is reported as the average of three test sample replications of Steps 1-7.

Optional Ingredients

A variety of skin benefit agents may be included to improve after feel properties. Advantageously these substances will be available as anhydrous dry powders upon the outermost layers of article or alternatively deposited in the second layer webs along with the effervescent materials. Within the skin benefit agent scope are several categories of materials including but not necessarily limited to skin treatment composition comprising at least one agent selected from the group consisting of conditioning component, vitamins, zeolites, peptides, sunscreen actives, terpene alcohols, desquamation actives, anti-acne actives, anti-wrinkle actives, anti-atrophy actives, anti-oxidants, flavanoids, anti-inflammatory agents, anti-cellulite agents, topical anesthetics, tanning actives, chelators, skin lightening agents, antimicrobial actives, anti-fungal actives, skin soothing actives, skin healing actives, skin moisturizing actives, cosmetic actives and mixtures thereof. Amounts of the skin benefit agents may range from about 0.001% to about 30%, alternatively from about 0.01% to about 20%, alternatively from about 0.05% to about 10% and finally from about 0.06% and about 5% by weight of the total composition.

1) Emollients may be in the form of natural or synthetic esters, silicone oils, hydrocarbons, starches, fatty acids and mixtures thereof. Typically the emollient may range in concentration from about 0.1% to about 35% by weight of the total composition.

The ester emollients useful in the present invention are selected from the group consisting of alkenyl or alkyl esters of fatty acids having 10 to 20 carbon atoms, including isoarachidyl neopentanoate, isononyl isonanonoate, oleyl myristate, oleyl stearate, and oleyl oleate and mixtures thereof; ether-esters, including fatty acid esters of ethoxylated fatty alcohols; polyhydric alcohol esters, including ethylene glycol mono and di-fatty acid ester, diethylene glycol mono- and di-fatty acid esters, polyethylene glycol (200-6000) mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters and mixtures thereof; wax esters, including beeswax, spermaceti, myristyl myristate, stearyl stearate, arachidyl behenate and mixtures thereof; sterols esters, including cholesterol fatty acid esters; triglycerides, including sunflower seed oil, maleated sunflower seed oil, borage seed oil, safflower oil and mixtures thereof as well as any combination of the preceding emollients.

Silicone oils may be divided into the volatile and nonvolatile variety. The term “volatile” as used herein refers to those materials that have a measurable vapor pressure at ambient temperature. Volatile silicone oils are alternatively chosen from cyclic or linear polydimethylsiloxanes containing from 3 to 9, alternatively from 4 to 5, silicon atoms. Linear volatile silicone materials generally have viscosities less than about 5 centistokes at 25° C. while cyclic materials typically have viscosities of less than about 10 centistokes. Nonvolatile silicone oils useful as an emollient material include polyalkyl siloxanes, polyalkylaryl siloxanes and polyether siloxane copolymers. The essentially non-volatile polyalkyl siloxanes useful herein include, for example, polydimethyl siloxanes with viscosities of from about 5 to about 100,000 centistokes at 25° C. Among the preferred non-volatile emollients useful in the present compositions are the polydimethyl siloxanes having viscosities from about 10 to about 400 centistokes at 25° C.

Hydrocarbons suitable as emollients include petrolatum, mineral oil, isoparaffins and hydrocarbon waxes such as polyethylene and mixtures thereof. Starches are also suitable emollients. Typical of this class is tapioca and arabinogalactan. Fatty acids may also be suitable as emollients. The fatty acids normally have from 10 to 30 carbon atoms. Illustrative of this category are pelargonic, lauric, myristic, palmitic, stearic, isostearic, hydroxystearic, oleic, linoleic, riconleic, arachidic, behenic and erucic acids and combinations thereof.

2) Antiaging actives are also useful as skin benefit agents. Included within this category are vitamins, retinoids and combinations thereof. Amounts of these materials may range from about 0.001 to about 20% by weight of the total composition. Suitable vitamins include ascorbic acid, Vitamin B.sub.6, Vitamin B.sub.12, tocopherol as well as salts and C.sub.1-C.sub.20 esters thereof. Suitable retinoids include retinoic acid as well as its C.sub.1-C.sub.22 esters and salts, retinol and C.sub.1-C.sub.22 fatty esters of retinol including retinyl linoleate.

Another class of antiaging actives are the alpha- and beta-hydroxycarboxylic acids and salts thereof. Representative of this group are glycolic acid, lactic acid, malic acid, hydroxyoctanoic acid and mixtures of these as well as their salts. Suitable salts are the alkalimetal, ammonium and C.sub.1-C.sub.10 alkanol ammonium salts.

3) Antibacterials and fungicidals may also be included as skin benefit agents. Representative of these categories are triclosan, tricloban, hexetidene, chlorhexadene, gluconates, zinc salts (e.g. zinc citrate and zinc phenolsulfonate) and mixtures thereof.

4) Skin lighteners may also be included under the skin benefit agents. Typical of this category are niacinamide, kojic acid, arbutin, vanillin, ferulic acid and esters thereof, resorcinol, hydroquinone, placental extract and mixtures thereof.

5) Sunscreens may also be included as skin benefit agents. Particularly preferred are such materials as ethylhexyl p-methoxycinnamate, available as Parsol® MCX, and benzophenone-3, also known as Oxybenzone. Inorganic sunscreen actives may be employed such as microfine titanium dioxide, polyethylene and various other polymers. Amounts of the sunscreen agents will generally range from 0.1 to 30%, alternatively from about 2% to about 20%, optimally from about 4% to about 10% by weight.

6) Adjunct functional agents may also be incorporated into compositions of the present invention. These include electrolytes, thickeners and mixtures thereof. Amounts of these substances may range from about 0.1% to about 20%, alternatively from about 0.3% to about 10%, optimally between about 0.5% to about 5% by weight of the total composition.

Electrolytes may be selected from alkali, alkaline earth or ammonium salts of phosphates, silicates, halides, sulphates and mixtures thereof. Typical phosphates are potassium polymetaphosphate, sodium tripolyphosphate, sodium tetrapyrophosphate, sodium or potassium pyrophosphate and sodium hexametaphosphate. Most preferred is potassium polymetaphosphate available as Lipothix 100B® which is a 70:30 mixture of potassium polymetaphosphate and sodium bicarbonate, available from Lipo Chemicals, Inc., Paterson, N.J. Preferred sulphates are the magnesium sulphates.

Thickeners that may improve after feel properties on skin include inorganic or organic substances. A particularly preferred inorganic thickener is sodium magnesium silicate commercially available as Optigel SH®. Organic thickeners include alginic acid as well as sodium and calcium alginates, sodium carboxymethyl cellulose, hydroxypropyl methylcellulose, hydroxypropyl cellulose, hydroxyethyl cellulose and combinations thereof. Most preferred is alginic acid commercially available as Kelacid® from Sud-Chemie Rheologicals, Louisville, Ky. Alginic acid is highly effective at removing the slimy feel associated with deposits of alkaline material which are not fully rinsed away from the skin. Amounts of the thickener may range from about 0.1 to about 20%.

Polysaccharides useful in this invention are dry solid anhydrous substances such as sucrose polyesters, sorbitol, sugars, (such as trehalose) starches, modified starches (e.g. aluminum octenyl succinate) and mixtures thereof.

Deposition aids may also be incorporated in compositions of the present invention. These assist in depositing skin benefit agents onto the skin surface. Particularly effective are cationic monomers and polymers for this purpose. Most preferred for purposes of this invention are cationic guar gums such as Jaguar C13S® which is guar hydroxypropyltrimonium chloride. Amounts of the deposition aid may range from about 0.01 to about 1%, alternatively from about 0.05% to about 0.5%, optimally from about 0.1% about 0.3% by weight.

Advantageously an emotive agent such as a fragrance and/or botanical extract are included with the effervescent cleansing composition. Fragrances and botanicals are often liquids. For this reason it is necessary to uniformly distribute and allow absorption of liquid components into the solid powder. One method of best achieving this is to spray these liquids onto the solids. Amounts of the fragrance and/or botanicals combined may be at levels from 0.1% to 3%, alternatively from 0.5% to 2%, optimally from 0.8% to 1.5% by weight of the total composition.

The terms “fragrance” and “botanical extract” are defined as mixtures of odoriferous components, optionally mixed with a suitable solvent diluent or carrier, which is employed to impart a desired odor. Particular preferred odoriferous components are cyclic and acyclic terpenes and terpenoids. These materials are based upon isoprene repeating units. Examples include alpha and beta pinene, myrcene, geranyl alcohol and acetate, camphene, di-limonene, alpha and beta phellandrene, tricyclene, terpinolene, allocimmane, geraniol, nerol, linanool, dihydrolinanool, citral, ionone, methyl ionone, citronellol, citronellal, alpha terpineol, beta terpineol, alpha fenchol, borneol, isoborneol, camphor, terpinen-1-ol, terpin-4ol, dihydroterpineol, methyl chavicol, anethole, 1,4 and 1,8 cineole, geranyl nitrile, isobornyl acetate, linalyl acetate, caryophyllene, alpha cedrene, guaiol, patchouli alcohol, alpha and beta santalol and mixtures thereof. Botanical extracts of particular use in the present invention include those extracted from yarrow, chamomile, jasmine, lavender, horse chestnut, sage, thyme, yucca, coltsfoot and mixtures thereof.

Preservatives can desirably be incorporated into the cosmetic compositions of this invention to protect against the growth of potentially harmful microorganisms. Particularly preferred preservatives are phenoxyethanol, methyl paraben, propyl paraben, imidazolidinyl urea, sodium dehydroacetate and benzyl alcohol. The preservatives should be selected having regard for the sue of the composition and possible incompatibilities between the preservatives and other ingredients in the emulsion. Preservatives are alternatively employed in amounts ranging from 0.01% to 2% by weight of the composition.

Colorants may also be included in compositions of the present invention. These substances may range from about 0.05 to about 5%, alternatively between 0.1 and 3% by weight.

Skin surfaces against which articles of the present invention are useful include face, body, scalp, axilla and even legs/feet. When the article is a foot cleanser, it would be advantageous for the article on one of its sides to be coarse while the second of the sheets may be soft and gentle. An abrasive non-woven flexible sheet in a foot cleanser product is useful for rubbing against calluses while the second sheet of the article remains smooth.

Manufacturing the Articles

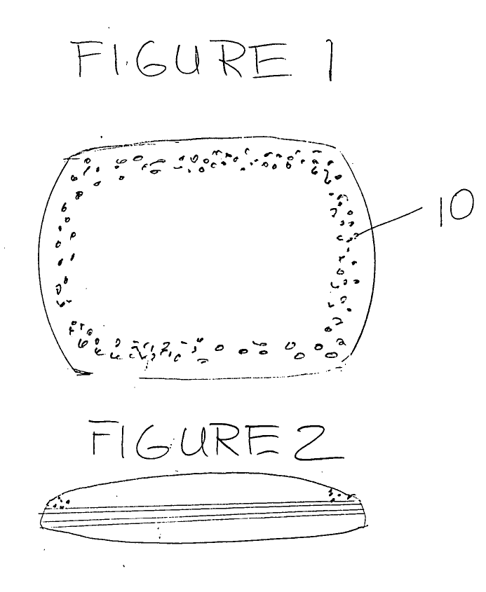

Articles according to the present invention may be formed in a variety of ways. An illustrative but certainly non-limiting example is as follows. Constituents of the effervescent composition (phase C in examples below) are placed into a dry mill or similar apparatus with a binder material and blended until a uniformly distributed powder results. The laminate comprises water-permeable layers whereby the effervescent composition is located between the webs making up the second layer as described above. The second layer, either made at the time or manufactured of the article or in advance, is superimposed on the first layer wherein the two layers proceed through a heated lamination nip to bond the layers together. In the case where additional layers are being added to the laminate, each layer may be added simultaneously or sequentially to the first and second layers. Any liquid fragrances may be sprayed onto any of the layers, usually exclusive of the batting layer during assembly of said laminate. Regardless of the number of layers, all layers are ultrasonically bonded as illustrated in FIG. 1, number 10. Heat bonding may be employed as an alternative to ultrasonic bonding. Thread stitching, glue application or other closure mechanisms may also be utilized. Said article is bonded in such a manner to provide a particular shape for said article including, but not limited to the shape as illustrated in FIGS. 1 and 2.

Upon manufacture of the articles described above, a plurality of the articles are inserted into packaging and is sealed. The package also has a closure that can be repeatedly opened and closed to gain access to the supply of articles in the package. Such packaging is disclosed in U.S. Pat. No. 5,050,763, to Christensson, issued Sep. 24, 1999. After purchase of the product, the consumer opens the closure, removes the seal and removes a desired number of the cleansing articles found therein. The consumer closes the closure for storing the remaining non-selected articles until their next use wherein the closure is again opened to provide access to the articles in the package. Ideally, the closure is one that seals the package even after removing the seal upon the initial use of the article. This way the articles are protected from moisture and humidity sufficiently enough to prevent unwanted reactivity of the effervescent composition of the article prior to use. Furthermore, the package may comprise vents to allow escape of any residual gas from within the sealed package.

Except in the operating and comparative examples, or where otherwise explicitly indicated, all numbers in this description indicating amounts of material are to be understood as modified by the word “about”.

The term “comprising” is meant not to be limiting to any subsequently stated elements but rather to encompass non-specified elements of major or minor functional importance. In other words the listed steps, elements or options need not be exhaustive.

All measurements referred to herein are made at 25° C. unless otherwise specified. All publications, patent applications and issued patents mentioned herein are hereby incorporated in their entirety by reference. Citation of any reference is not an admission regarding any determination as to its availability as prior art to the claimed invention.

The following examples will more fully illustrate embodiments of this invention. All parts, percentages and proportions referred to herein and in the appended claims are by weight unless otherwise indicated.

### EXAMPLE 1

| Ingredient | Weight % |
|---|---|
| Phase A(@ 12 gsm × 2 layers) | |
| Glycerin | 2.21 |
| Tegobetaine FB | 1.46 |
| Plantaren 2000 N UP | 1.46 |
| Hamposyl L-30 | 1.46 |
| Butylene Glycol | 0.44 |
| Polyox WSR N3000 | 0.22 |
| Ucare Polymer JR30M | 0.11 |
| D-Panthenol | 0.089 |
| Emeressence 1160 | 0.066 |
| Benzyl Alcohol NF | 0.066 |
| Methyl Paraben | 0.055 |
| Propyl Paraben | 0.033 |
| Disodium EDTA | 0.021 |
| Grape Seed Extract | 0.0024 |
| Chamomile Extract | 0.0024 |
| Veragel 1:1 | 0.0024 |
| Vitamin E Acetate | 0.0024 |
| Phase B | |
| Super White Protopet | 0.81 |
| Phase C | |
| Sodium Bicarbonate | 50.33 |
| Citric Acid | 41.16 |
| Non-woven Materials | |
| Fourth layer | high loft batting |
| Third layer | HEF |
| Second layer | chemistry containing web |
| First layer | textured HEF |

An article of the present invention is made using materials as found in the table above. Using a slot coater, homogeneous Phase A is coated on to the surface of the first layer opposite the contacting surface of the first and second layers at a rate of 12 grams/meter². Phase A is also added on to the surface of third layer opposite the surface contacting surfaces of the second and third layers at a rate of 12 grams/meter². Phase B is added on to the surface of third layer at the surface opposite the contacting surface of the second and third layers at a rate of 3.5 grams/meter². Phase C was dry blended in an auger and laminated into place at the contacting surfaces of the web or webs forming the second layer at a rate of 390 grams/meter².

### EXAMPLE 2

| Ingredient | Weight % |
|---|---|
| Phase A(@ 12 gsm × 2 layers) | |
| Glycerin | 2.21 |
| Hydroxysultaine | 1.46 |
| Plantaren 2000 N UP | 1.46 |
| Hamposyl L-30 | 1.46 |
| Butylene Glycol | 0.44 |
| Polyox WSR N3000 | 0.22 |
| Ucare Polymer JR30M | 0.11 |
| D-Panthenol | 0.089 |
| Emeressence 1160 | 0.066 |
| Benzyl Alcohol NF | 0.066 |
| Methyl Paraben | 0.055 |
| Propyl Paraben | 0.033 |
| Disodium EDTA | 0.021 |
| Grape Seed Extract | 0.0024 |
| Chamomile Extract | 0.0024 |
| Veragel 1:1 | 0.0024 |
| Vitamin E Acetate | 0.0024 |
| Fragrance | 0.0024 |
| Phase B | |
| Super White Protopet | 0.81 |
| Phase C | |
| Sodium Bicarbonate | 50.33 |
| Citric Acid | 41.16 |
| Non-woven Materials | |
| Third layer | polypropylene spunbond |
| Second layer | chemistry containing web |
| First layer | textured HEF |

An article of the present invention is made using materials as found in the table above. Using a slot coater, homogeneous Phase A is coated on to the surface of the first layer opposite the contacting surface of the first and second layers at a rate of 12 grams/meter². Phase A is also added on to the surface of third layer opposite the surface contacting the second and third layers at a rate of 12 grams/meter². Phase B is added on to the surface of fourth layer at the surface opposite the contacting surface of the third and fourth layers at a rate of 3.5 grams/meter². Phase C was dry blended in an auger and laminated into place at the contacting surfaces of the web or webs forming the second layer at a rate of 390 grams/meter².

### EXAMPLE 3

| Ingredient | Weight % |
|---|---|
| Phase A(@ 12 gsm × 2 layers) | |
| Glycerin | 2.21 |
| Hydroxysultaine | 1.46 |
| Plantaren 2000 N UP | 1.46 |
| Hamposyl L-30 | 1.46 |
| Butylene Glycol | 0.44 |
| Polyox WSR N3000 | 0.22 |
| Ucare Polymer JR30M | 0.11 |
| D-Panthenol | 0.089 |
| Emeressence 1160 | 0.066 |
| Benzyl Alcohol NF | 0.066 |
| Methyl Paraben | 0.055 |
| Propyl Paraben | 0.033 |
| Disodium EDTA | 0.021 |
| Grape Seed Extract | 0.0024 |
| Chamomile Extract | 0.0024 |
| Veragel 1:1 | 0.0024 |
| Vitamin E Acetate | 0.0024 |
| Fragrance | 0.0024 |
| Phase B | |
| Super White Protopet | 0.81 |
| Phase C | |
| Sodium Bicarbonate | 50.33 |
| Citric Acid | 41.16 |
| Non-woven Materials | |
| First layer | polypropylene spunbond |
| Second layer | chemistry containing web |

An article of the present invention is made using materials as found in the table above. Using a slot coater, homogeneous Phase A is coated on to the surface of the first layer opposite the contacting surface of the first and second layers at a rate of 12 grams/meter². Phase A is also added on to the surface of the second layer opposite the surface contacting the first and second layers at a rate of 12 grams/meter². Phase B is also added on to the surface of second layer at the surface opposite the contacting surface of the first and second layers at a rate of 3.5 grams/meter². Phase C was dry blended in an auger and laminated into place at the contacting surfaces of the web or webs forming the second layer at a rate of 390 grams/meter².

The foregoing description and examples illustrate selected embodiments of the present invention. In light thereof variations and modifications will be suggested to one skilled in the art, all of which are within the spirit and purview of this invention.

All documents cited in the Detailed Description of the Invention are, are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. An article for cleansing body surfaces, the article comprising:
a) an effervescent composition comprising solid alkaline and acidic materials; and
b) a liquid-permeable laminate comprising a plurality of layers, said laminate having a first layer comprising a web and a second layer, in contact with said first layer, comprising a plurality of webs wherein said effervescent composition is present between the webs comprising said second layer.

2. The cosmetic article of claim 1 wherein said web of said first layer comprises a lathering surfactant distributed over said first layer on the surface of said layer's web opposite the contact surfaces of said first layer and said second layer.

3. The cosmetic article of claim 2 where the lathering surfactant is selected from the group consisting of anionic lathering surfactants, nonionic lather surfactants, amphoteric lathering surfactants and mixtures thereof.

4. The cosmetic article of claim 3 where the lathering surfactant is at a level from about 0.01% to 1500%, by weight of said article.

5. The cosmetic article of claim 4 wherein the anionic lathering surfactants are selected from the group consisting of sarcosinates, sulfates, isethionates, taurates, phosphates, lactylates, glutamates, and mixtures thereof.

6. The cosmetic article of claim 5 wherein said second layer's webs are affixed to one another at the contacting surfaces of the webs by attachment means selected from the group consisting of bonding agents, sonic welding, heat fusing, mechanical compression and combinations thereof.

7. The cosmetic article of claim 6 wherein said bonding agents are selected from the group consisting of polyethylene glycol, polyethylene and mixtures thereof.

8. The cosmetic article of claim 7 wherein said article has a third layer attached to said second layer at the surface of said second layer opposite the contacting surface of said first layer and said second layer.

9. The cosmetic article of claim 8 wherein the article's layers are bonded together about the perimeter of said article.

10. The cosmetic article of claim 9 wherein said effervescing composition is dry and comprises from about 1 to about 80% of the total weight of the effervescing composition an alkaline material selected from the group consisting of anhydrous salts of carbonates and bicarbonates; alkaline peroxides and azides and mixtures thereof.

11. The cosmetic article of claim 10 wherein said alkaline material is selected from the group consisting sodium bicarbonate, potassium bicarbonate, sodium carbonate, potassium carbonate and mixtures thereof.

12. The cosmetic article of claim 11 wherein said effervescing composition comprises from about 0.5% to about 80% of the total weight of the effervescing composition an acidic material is selected from the group consisting of C₂ to C₂₀ organic mono- and poly-carboxylic acids; C₂ to C₂₀ organophosphorus acids; C₂ to C₂₀ organosulfur acids, toluene sulfonic acid; peroxides, lactones and mixtures thereof.

13. The cosmetic article of claim 12 wherein said hydroxycarboxylic acids are selected from the group consisting of adipic, glutaric, succinic, tartaric, malic, maleic, lactic, salicylic, citric acids and mixtures thereof.

14. An article for cleansing body surfaces, the article comprising:
a) an effervescent composition comprising solid alkaline and acidic materials; and
b) a liquid-permeable laminate comprising a plurality of layers, said laminate's first layer comprising a web and a second layer in contact with said first layer, wherein said second layer comprises a two webs wherein said effervescent composition is provided to the contacting surfaces of said webs comprising said second layer; and
c) a third layer contacting said second layer at the surface of said second layer opposite that contacting the surface of said first layer.

15. The cosmetic article of claim 14 wherein said web of said first layer comprises a lathering surfactant distributed over said first layer on the surface of said layer's web opposite the contact surfaces of said first layer and said second layer.

16. The cosmetic article of claim 15 further comprising a skin treatment agent at a level from about 0.001 to about 30% by weight of the article, said skin treatment agent located on the surface of said third layer opposite said surface contacting said second layer.

17. The cosmetic article of claim 16 wherein said skin treatment agent is selected from the group consisting of conditioning component, vitamins, zeolites, peptides, sunscreen actives, terpene alcohols, desquamation actives, anti-acne actives, anti-wrinkle actives, anti-atrophy actives, anti-oxidants, flavanoids, anti-inflammatory agents, anti-cellulite agents, topical anesthetics, tanning actives, chelators, skin lightening agents, antimicrobial actives, anti-fungal actives, skin soothing actives, skin healing actives, skin moisturizing actives, cosmetic actives and mixtures thereof.

18. The cosmetic article of claim 17 further comprising from about 0.010% to about 35% by weight of article of an emollient.

19. The cosmetic article of claim 18 wherein said emollients are selected from the group consisting of natural or synthetic esters, silicone oils, hydrocarbons, starches, fatty acids, triglycerides, waxes and mixtures thereof.

20. The cosmetic article of claim 19 wherein said third layer has a lathering surfactant upon its surface opposite its surface contacting said second layer.

21. An article for cleansing body surfaces, the article comprising:
a) an effervescent composition comprising solid alkaline and acidic materials; and
b) a liquid-permeable laminate comprising a plurality of layers, said laminate having a first layer comprising a web and a second layer in contact with said first layer, wherein said second layer comprises two webs wherein said effervescent composition is present between said first and second web of said second layer;
c) a third layer contacting said second layer at the surface of said second layer opposite that contacting said first layer; and
d) a fourth layer contacting said third layer at the surface of said third layer opposite that contacting the second layer wherein said fourth layer comprises a high loft batting material.

22. The cosmetic article of claim 21 wherein said web of said first layer comprises a lathering surfactant distributed over said first layer on the surface of said layer's web opposite the contact surfaces of said first layer and said second layer, and distributed over said third layer on the surface of said layer's web opposite the contact surfaces of said second layer and said third layer.

23. The cosmetic article of claim 21 wherein said fourth layer has a lathering surfactant over the surface of said high loft batting material.

24. A kit for the use of cosmetic article for cleansing body surfaces, said kit comprising:
a) an effervescent composition comprising solid alkaline and acidic materials;
b) a cleansing article comprising a liquid-permeable laminate comprising a plurality of layers, said laminate's first layer comprising a web and a second layer in contact with said first layer, wherein said second layer comprises two webs wherein said effervescent composition is provided on to the contacting surface of said webs comprising said second layer; and
c) a package for storage of a plurality of said cleansing articles, said package having a removable seal and a closure, wherein upon opening the closure and removing the seal, a desired number of cleansing articles are removed from said package wherein the closure is closed to store the remaining non-selected articles in the package.

25. The kit according to claim 24 wherein the package comprises a hinged closure and said package comprises vents to allow escape of any residual gas from within the sealed package.
